(19) United States
(12) Patent Application Publication
(10) Pub. No.: US 2026/0266786 A1
(43) Pub. Date: Sep. 10, 2026

(54) SULFUR DETECTOR AND FUEL PROCESSING SYSTEM INCLUDING THE SAME

(71) Applicant: Bloom Energy Corporation, San Jose, CA (US)

(72) Inventors: Madhura R. REWATKAR, Mumbai (IN); Shraddesh MALVIYA, Mumbai (IN); Mohammad KHAN, Mumbai (IN); Amol K. PATIL, Mumbai (IN); Raja PUJARI, Mumbai (IN); Ilina PUNJA, Los Angeles, CA (US); Srikanth RANGANATHAN, Fremont, CA (US); Brandon SNOW, Palo Alto, CA (US)

(21) Appl. No.: 19/534,002

(22) Filed: Feb. 9, 2026

Related U.S. Application Data
(60) Provisional application No. 63/768,631, filed on Mar. 7, 2025.

(51) Int. Cl. G01N 33/00; B01D 53/86; C01B 17/16; G01N 27/12; H01M 8/0444; H01M 8/04746; H01M 8/0662; H01M 8/12

(52) U.S. Cl. CPC G01N 33/0013; B01D 53/8603; C01B 17/165; G01N 27/12; H01M 8/04447; H01M 8/04753; H01M 8/0675; B01D 2255/2092; B01D 2257/308; B01D 2258/05; H01M 2008/1293

(57) ABSTRACT

A sulfur detector may include a sulfur compound conversion unit configured to convert a sulfur-containing compound into hydrogen sulfide, and a sensor unit configured to detect the hydrogen sulfide.

### FIELD

Various embodiments relate to a sulfur detector and a fuel processing system including the sulfur detector, and in particular, to a sulfur detector that detects a hydrolyzed sulfur-containing compound in a fuel provided to a fuel cell system.

### BACKGROUND

Fuel cells, such as solid oxide fuel cells (SOFC's), are electrochemical devices which can convert energy stored in fuels to electrical energy with high efficiencies. High temperature fuel cells include solid oxide and molten carbonate fuel cells. These fuel cells may operate using hydrogen and/or hydrocarbon fuels. There are classes of fuel cells, such as the solid oxide regenerative fuel cells, that also allow reversed operation, such that oxidized fuel can be reduced back to unoxidized fuel using electrical energy as an input.

The reliability of fuel cell systems, such as SOFC systems, is impacted by the presence and concentration of contaminants in the fuel stream. Contaminants, such as sulfur and sulfur compounds, may degrade the fuel cell stack's performance resulting in decreased efficiencies and costly replacement for impacted parts. Accordingly, when using a hydrocarbon fuel containing sulfur species, there is a need for fuel cell systems to utilize a desulfurization system to remove sulfur from the hydrocarbon fuel.

### SUMMARY

According to various embodiments of the present disclosure, a sulfur detector may include a sulfur compound conversion unit configured to convert a sulfur-containing compound into hydrogen sulfide (H₂S); and a sensor unit configured to detect the H₂S.

According to various embodiments of the present disclosure, a method of detecting a sulfur-containing compound in a fuel may include converting a sulfur-containing compound in the fuel into hydrogen sulfide (H₂S); and detecting the H₂S.

### BRIEF DESCRIPTION OF THE DRAWINGS

The accompanying drawings, which are incorporated herein and constitute part of this specification, illustrate example embodiments of the invention, and together with the general description given above and the detailed description given below, serve to explain the features of the invention.

FIG. 1A is a schematic representation of a SOFC system, according to various embodiments of the present disclosure, and FIG. 1B is a perspective view of a SOFC system illustrating components that could be included in the SOFC system of FIG. 1A, according to various embodiments of the present disclosure.

FIG. 2A is a schematic view of the desulfurization system of FIG. 1A, according to first and second embodiments of the present disclosure.

FIG. 2B is cross-sectional view of a reaction vessel of that may be included in the desulfurization system of FIG. 2A, according to the first embodiment of the present disclosure.

FIG. 3 is a schematic representation of a sulfur detector that may be used with the desulfurization system of FIG. 2A, according to one or more embodiments of the present disclosure.

FIG. 4 is a perspective view of the sensor unit that may be utilized in the sulfur detector of FIG. 3, according to one or more embodiments of the present disclosure.

FIG. 5 is a schematic representation of an alternative configuration of the sulfur detector, according to an alternative embodiment of the present disclosure.

FIG. 6 is a schematic representation of another alternative sulfur detector, according to another alternative embodiment of the present disclosure.

### DETAILED DESCRIPTION

The various embodiments will be described in detail with reference to the accompanying drawings. Wherever possible, the same reference numbers will be used throughout the drawings to refer to the same or like parts. References made to particular examples and implementations are for illustrative purposes and are not intended to limit the scope of the invention or the claims.

Ranges can be expressed herein as from “about” one particular value, and/or to “about” another particular value. When such a range is expressed, examples include from the one particular value and/or to and including the other particular value. In some embodiments, a value of “about X” may include values of +/−1% X. It will be further understood that the endpoints of each of the ranges are significant both in relation to the other endpoint, and independently of the other endpoint.

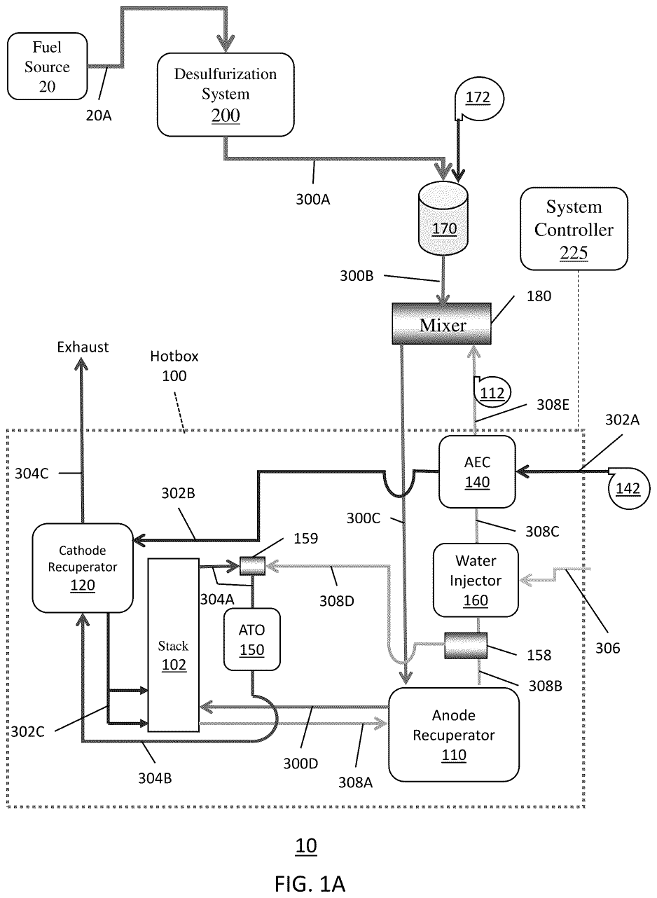

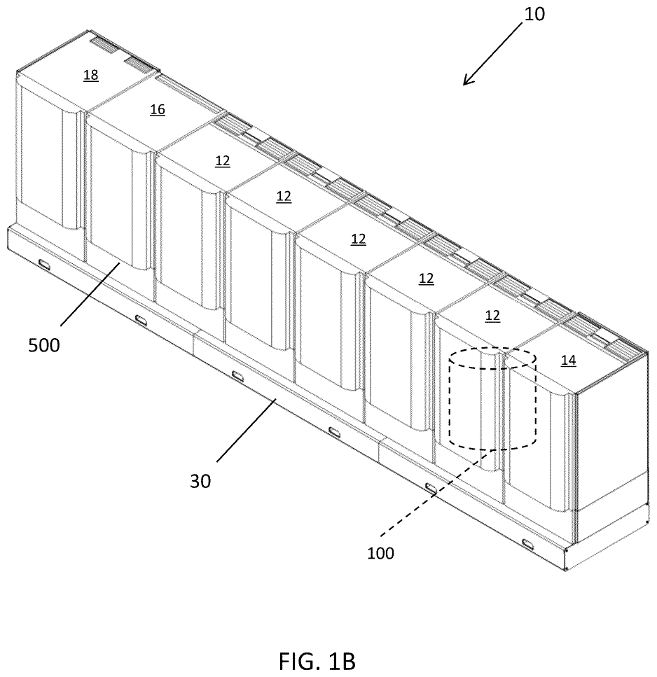

FIG. 1A is a schematic representation of a SOFC system 10, according to various embodiments of the present disclosure. Referring to FIG. 1A, the system 10 includes a hotbox 100 and various components disposed therein or adjacent thereto. The hotbox 100 may contain one or more fuel cell stacks 102, which may include solid oxide fuel cells separated by interconnects.

The hotbox 100 may also contain an anode recuperator heat exchanger 110, a cathode recuperator heat exchanger 120, an anode tail gas oxidizer (ATO) 150, an anode exhaust cooler heat exchanger (AEC) 140, a splitter 158, a vortex generator 159, and a water injector 160. The system 10 may also include a catalytic partial oxidation (CPOx) reactor 170, a mixer 180, a CPOx blower 172 (e.g., air blower), a main air blower 142 (e.g., system blower), and an anode recycle blower 112, which may be disposed outside of the hotbox 100. However, the present disclosure is not limited to any particular location for each of the components with respect to the hotbox 100.

A hydrocarbon fuel may be provided to the CPOx reactor 170 by fuel conduit 300A.

The CPOx blower 172 may provide air to the CPOx reactor 170 during system start-up. The fuel and/or air may then be provided to the mixer 180 by fuel conduit 300B. Fuel (e.g., the fuel inlet stream) flows from the mixer 180 to the anode recuperator 110 through fuel conduit 300C. The fuel is heated in the anode recuperator 110 by a portion of the fuel exhaust and the fuel then flows from the anode recuperator 110 to the stack 102 through fuel conduit 300D.

The main air blower 142 may be configured to provide an air stream (e.g., air inlet stream) to the anode exhaust cooler 140 through air conduit 302A. Air flows from the anode exhaust cooler 140 to the cathode recuperator 120 through air conduit 302B. The air is heated by the ATO exhaust in the cathode recuperator 120. The air flows from the cathode recuperator 120 to the stack 102 through air conduit 302C.

An anode exhaust stream (e.g., the fuel exhaust stream) generated in the stack 102 is provided to the anode recuperator 110 through anode exhaust conduit 308A. The anode exhaust may contain unreacted fuel and may also be referred to herein as fuel exhaust. The anode exhaust may be provided from the anode recuperator 110 to the splitter 158 by anode exhaust conduit 308B. A first portion of the anode exhaust may be provided from the splitter 158 to the anode exhaust cooler 140 through the water injector 160 and the anode exhaust conduit 308C. A second portion of the anode exhaust can be provided from the splitter 158 to the ATO 150 through the anode exhaust conduit 308D. The first portion of the anode exhaust heats the air inlet stream in the anode exhaust cooler 140 and may then be provided from the anode exhaust cooler 140 to the mixer 180 through the anode exhaust conduit 308E.

The anode recycle blower 112 may be configured to move anode exhaust though anode exhaust conduit 308E, as discussed below.

Cathode exhaust generated in the stack 102 flows to the ATO 150 through exhaust conduit 304A. A vortex generator 159 may be disposed in exhaust conduit 304A and may be configured to swirl the cathode exhaust. The anode exhaust conduit 308D may be fluidly connected to the vortex generator 159 or to the cathode exhaust conduit 304A or the ATO 150 downstream of the vortex generator 159. The swirled cathode exhaust may mix with the second portion of the anode exhaust provided by the splitter 158 before being provided to the ATO 150. The mixture may be oxidized in the ATO 150 to generate an ATO exhaust. The ATO exhaust flows from the ATO 150 to the cathode recuperator 120 through exhaust conduit 304B. Exhaust flows from the cathode recuperator and out of the hotbox 100 through exhaust conduit 304C.

Water flows from a water source, such as a water tank or a water pipe, to the water injector 160 through water conduit 306. The water injector 160 injects water directly into a first portion of the anode exhaust provided in anode exhaust conduit 308C. Heat from the first portion of the anode exhaust (also referred to as a recycled anode exhaust stream) provided in anode exhaust conduit 308C vaporizes the water to generate steam. The steam mixes with the anode exhaust, and the resultant mixture is provided to the anode exhaust cooler 140. The mixture is then provided from the anode exhaust cooler 140 to the mixer 180 through the anode exhaust conduit 308E. The mixer 180 is configured to mix the steam and the first portion of the anode exhaust with fresh fuel (i.e., the fuel inlet stream). This humidified fuel mixture may then be heated in the anode recuperator 110 by the anode exhaust, before being provided to the stack 102. The system 10 may also include one or more fuel reforming catalysts located inside and/or downstream of the anode recuperator 110. The reforming catalyst(s) reform the humidified fuel mixture before it is provided to the stack 102.

The system 10 may further a system controller 225 configured to control various elements of the system 10. The controller 225 may include a central processing unit configured to execute stored instructions. For example, the controller 225 may be configured to control fuel and/or air flow through the system 10, according to fuel composition data.

FIG. 1B is a perspective view of additional components that can be incorporated into SOFC system 10, according to various embodiments of the present disclosure. Referring to FIG. 1B, system 10 may have a modular system layout. System 10 may contain modules and components described in U.S. Pat. Nos. 9,190,693, 9,755,263, 10,797,327 and 11,862,832, all of which are incorporated herein by reference in their entireties. The modular design of system 10 may provide flexible system installation and operation. The modular design also allows the use of available fuels and required voltages and frequencies which may vary by customer and/or by geographic region.

System 10 shown in FIG. 1B includes multiple power modules 12, an optional accessory module 14, a fuel processing module (FPM) 16, and a power conditioning (i.e., electrical output) module 18. Each module 12, 14, 16, 18 may comprise a respective cabinet (e.g., a housing, such as a metal housing) having a respective door to access the modules 12, 14, 16, 18. For example, the FPM 16 comprises the FPM cabinet 500, as shown in FIG. 1B. Fuel cell stacks or columns (e.g., one or more stacks 102) are located in the power modules 12 inside of hot boxes 100 (i.e., thermally insulated containers) located in the power module cabinets behind the doors.

System 10 may be disposed on a skid 30 that supports the modules 12, 14, 16, 18.

The skid 30 may include an upper surface (i.e., a deck) that rests upon rails that are connected to the deck. The skid 30 may be configured to enable quick deployments and/or temporary deployments of system 10 and may reduce installation costs and cycle times.

While one row of five power modules 12 is shown in FIG. 1B, the system 10 may comprise any number of power modules 12 (e.g., 1 to 20, such as 4 to 12 power modules) and any number of rows of power modules 12, such as two or more rows (e.g., two to ten rows).

Each power module 12 is configured to house at least one hotbox 100. Each hotbox 100 contains one or more stacks or columns of electrochemical cells (e.g., fuel cells), such as one or more stacks or columns of solid oxide fuel cells having a ceramic oxide electrolyte separated by conductive interconnect plates. Other fuel cell types, such as PEM, molten carbonate, phosphoric acid, etc. may also be used.

The system 10 may include or be fluidly connected to a desulfurization system 200 (also referred to as desulfurization reactor 200). The desulfurization system 200 may receive a hydrocarbon fuel, such as natural gas, methane, propane, butane, biogas, etc., from a fuel source 20, such as a fuel tank, a fuel line, or a biogas generation site, via fuel source conduit 20A.

The system 200 may be configured to remove sulfur species (e.g., sulfur and/or sulfur compounds) from the fuel in order to provide desulfurized fuel to the system 10 (e.g., to the CPOx reactor 170) via the fuel conduit 300A. In some embodiments, the desulfurization system 200 may be configured to provide desulfurized fuel to multiple fuel cell systems 10.

In some embodiments, the fuel source 20 may be a biogas site including a fuel compressor. Compression of the biogas may increase the temperature of the fuel provided to the desulfurization system 200, which may increase sulfur removal rates.

Biogas is a renewable hydrocarbon fuel that can be produced from the anaerobic breakdown of organic raw materials, such as agricultural waste, manure, municipal waste, plant material, sewage, green waste, or food waste. Biogas may include a mixture of different gases. For example, biogas may include 50 to 80 volume % methane (CH₄), 20 to 50 volume % carbon dioxide (CO₂), and relatively small amounts (e.g., less than 5 volume %) of other components, such as sulfur species, moisture (e.g., water vapor), nitrogen, hydrogen, oxygen, carbon monoxide, siloxanes, ammonia, etc.

### Desulfurization Systems

Sulfur species are known to damage and deactivate fuel cell catalysts (e.g., anode electrode catalysts). Sulfur species include elemental sulfur and its compounds, such as non-metal sulfides, including hydrogen sulfide (H₂S), carbonyl sulfide (COS) and carbon disulfide CS₂, and organic sulfur (i.e., organosulfur) compounds, such as mercaptans, thiophenes, sulfides, and disulfides. Biogas may contain relatively high amounts of carbon-sulfur compounds, such as COS and CS₂, which may be particularly difficult to remove from gas streams using sulfur species sorption media (generally designed for H₂S removal primarily). Desulfurization technology may also require high operating temperatures (e.g., 200° C. or more, such as 300° C. or more) and/or high pressures to remove such carbon-sulfur compounds, and thus, may reduce system efficiency.

Accordingly, embodiments of the present disclosure include a mixed media desulfurization system comprising both a hydrolysis catalyst and a sulfur species sorbent. The hydrolysis catalyst converts COS and/or CS₂ to H₂S and CO₂ using residual moisture (e.g., water vapor) in the fuel stream, and the generated H₂S is then sequestered (i.e., captured by being adsorbed and/or absorbed) by the sulfur species sorbent. As used herein, a sulfur species sorbent includes adsorption and/or absorption materials which adsorb and/or absorb the sulfur species. An example of the sulfur species sorbent includes a sulfur species adsorption bed.

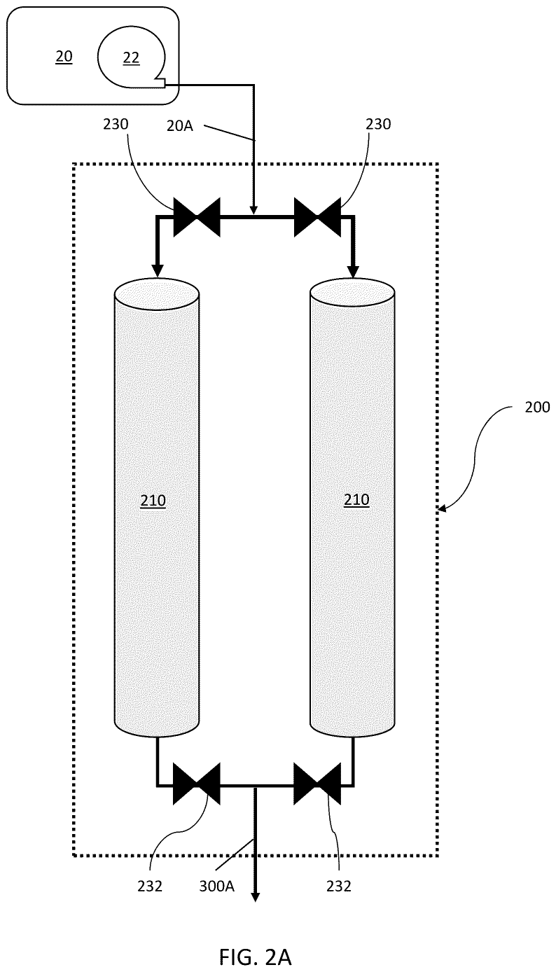

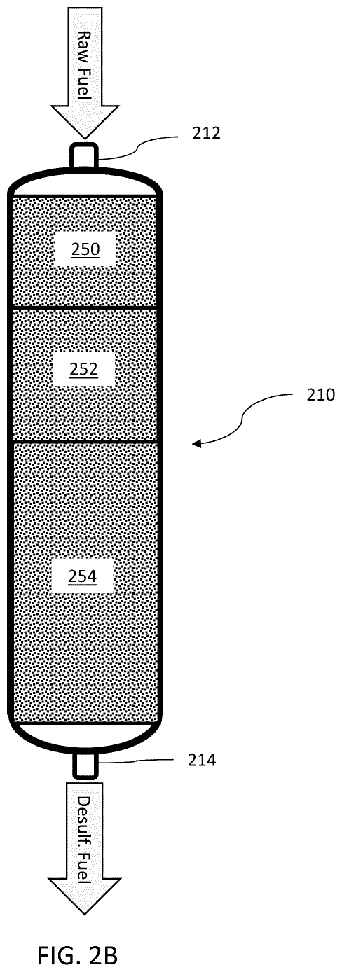

FIG. 2A is a schematic view of the desulfurization system 200 of FIG. 1A, according to various embodiments of the present disclosure, and FIG. 2B is cross-sectional view of a reaction (e.g., desulfurization) vessel 210 that may be included in the desulfurization system 200 of FIG. 2A. Referring to FIGS. 2A and 2B, the system 200 may include at least one reaction vessel 210 containing a desulfurization catalyst and/or sorbent configured to desulfurize a relatively low temperature fuel stream, such as a fuel stream having a temperature of 200° C. or less, such as a temperature of 25° C. to 75° C., for example a temperature of 50° C. to 65° C.

As shown in FIG. 2A, the system 200 may include at least two reaction vessels 210, inlet valves 230, and outlet valves 232. The system 200 may be configured to receive the fuel (e.g., the raw, sulfur species containing fuel inlet stream) from the fuel source 20 via the fuel source conduit 20A and may output desulfurized fuel (i.e., the desulfurized fuel inlet stream) to the fuel conduit 300A. In some embodiments, the system 200 may be configured to receive raw fuel from a compressor 22 of the fuel source 20. For example, the fuel source 20 may be a biogas generation site, and the compressor 22 can be an on-site biogas compressor. As such, the system 200 may be configured to operate using raw fuel heated (e.g., to a temperature of 50° C. to 65° C.) only by compression at a fuel source 20 site. Alternatively, the fuel source 20 may be a hydrocarbon fuel storage vessel or line, such as a biogas or natural gas storage vessel or line.

The inlet valves 230 and the outlet valves 232 may be configured to selectively control the raw fuel flow to the vessels 210. For example, the raw fuel may be selectively provided to one of the vessels 210, while no raw fuel is provided to the other vessel 210 to allow for servicing and/or replacement of the mixed media in the other vessel 210.

As shown in FIG. 2B, each vessel 210 may be a tube or conduit having an inlet 212 configured to receive raw fuel and an outlet 214 configured to output desulfurized fuel. The vessel 210 may be formed of a metal or a metal alloy, such as carbon steel, stainless steel or the like. The outer walls of the vessel 210 may be optionally covered by thermal insulation (not shown). The vessel 210 may include different types of sulfur catalysts and/or sorbents, which may be serially arranged in beds configured to sequentially receive the raw fuel provided to the vessel 210. For example, the vessel 210 may include a hydrolysis catalyst bed 250, a first sorption bed 252, and a second sorption bed 254. The first sorption bed 252 and the second sorption bed 254 may comprise sulfur species adsorption beds containing different sulfur species adsorption materials.

The first sorption bed 252 may be disposed downstream of the hydrolysis bed 250, and the second sorption bed 254 may be disposed downstream of the first sorption bed 252, with respect to a fuel flow direction through the vessel 210. Thus, the first sorption bed 252 is located between the hydrolysis bed 250 and the second sorption bed 254.

The hydrolysis bed 250 may be configured to receive the raw fuel from the inlet 212. The hydrolysis bed 250 may include a sulfur hydrolysis catalyst configured to hydrolyze carbon-sulfur species, such as COS and/or CS₂, to produce hydrogen sulfide and carbon dioxide, using water present in the raw fuel. Alternatively, if the raw fuel lacks sufficient water for the hydrolysis reaction, then water may be added to the fuel source 20 or the fuel source conduit 20A upstream of the inlet 212.

The hydrolysis catalyst may be an ambient hydrolysis catalyst having an operating temperature ranging from about 10° C. to about 200° C., such as a temperature ranging from about 20° C. to about 150° C., or from about 22° C. to about 65° C. The hydrolysis catalyst may be configured to hydrolyze sulfur species in fuels, such as raw biogas fuel, having a moisture content of up to 5000 ppm, such as from about 100 ppm to about 4500 ppm, including from about 200 ppm to about 3000 ppm. For example, the sulfur hydrolysis catalyst may hydrolyze COS using residual water vapor in the raw fuel to form H₂S and carbon dioxide using the hydrolysis reaction (i.e., COS+H₂O->CO₂+H₂S) at a temperature below 100° C., such as from about 22° C. to about 65° C.

In some embodiments, the hydrolysis bed 250 may include any suitable hydrolysis catalyst, such as metal oxide catalysts, mixed metal oxide catalysts, metal-metal oxide catalysts, bimetallic metal oxide catalysts, combinations thereof, or the like, which may be supported or unsupported and may be promoted or unpromoted. For example, suitable hydrolysis catalysts may include alumina (Al₂O₃), titania (TiO₂), zirconia (ZrO₂), ceria (CeO₂), silica (SiO₂), aluminosilicates, Co—Mo/Al₂O₃, Ni—Mo/Al₂O₃, other refractory metal oxide material catalysts, or combinations thereof. For example, adding TiO₂ to activated Al₂O₃ may provide a lower operating temperature than activated Al₂O₃ alone. The hydrolysis catalyst may be in the form of spheres or extruded pellets, which may be pressed together or contained in a porous bag or container. Suitable metal promotors may include Co, Cr, Fe, Ni, Mo, or the like.

The first sorption bed 252 may include a first desulfurization material configured to sequester (e.g., adsorb and/or absorb) organic sulfur compounds, such as mercaptans, thiophenes, sulfides, disulfides, and/or other trace contaminant species from fuel output from the hydrolysis bed 250. The first desulfurization material may be an ambient temperature sorption material. For example, the first desulfurization material may have an operating temperature ranging from about 15° C. to about 80° C., such as from about 20° C. to about 70° C., or from about 22° C. to about 66° C. The first desulfurization material may be configured to sorb sulfur species, such as organosulfur species, from fuels having a moisture content of up to about 5000 ppm, such as from about 100 ppm to about 4500 ppm, including from about 200 ppm to about 4000 ppm.

The first sorption bed 252 may include one or more metal oxide desulfurization materials and optionally one or more metal promotors disposed on a sorbent support. For example, the first sorption bed 252 may include CuO, Fe₂O₃, MnO₂, and ZnO desulfurization materials disposed on a support, such as an activated carbon sorbent support. In one embodiment, the material and/or a promoter may also be embedded in the support material.

The second sorption bed 254 may include a second desulfurization material different from the first desulfurization material. The second desulfurization material is configured to sequester non-metal sulfur compounds, such as H₂S, CS₂, and/or COS, from fuel received from the first sorption bed 252. The second desulfurization material may be an ambient temperature sorption material. For example, the second desulfurization material may have an operating temperature ranging from about 15° C. to about 90° C., such as from about 20° C. to about 85° C., or from about 22° C. to about 80° C. The second desulfurization material may be configured to sorb sulfur species from fuels having a moisture content of up to about 5000 ppm, such as from about 100 ppm to about 4500 ppm, including from about 200 ppm to about 4000 ppm.

For example, the second sorption bed 254 may include a copper oxide (e.g., CuO) and manganese oxide (e.g., MnO₂) based desulfurization materials having a high sorption capacity for both hydrogen sulfide, COS and organosulfur compounds, such as mercaptans, thiophenes, etc. The oxide materials may be disposed on a support, such as an activated carbon, zinc oxide or AlOₓ support. The second desulfurization material may be in the form of spheres or extruded pellets, which may be pressed together or contained in a porous bag or container. In one embodiment, the sorption material and/or a promoter may also be embedded in the support structure.

However, the present disclosure is not limited to the above catalysts and/or sorbents. In various embodiments, a number of different materials may be used for sequestering sulfur compounds. For example, activated carbon has a high capacity for ethyl mercaptans, manganese oxide is effective for dimethyl sulfoxide removal, and zinc oxide can be used to remove hydrogen sulfide. Other materials that may be used in desulfurization processes include copper/zinc oxides, nickel-based sorbents, nickel oxides, zeolites, and molecular sieves, among others. Nickel sorbents may be used to sorb many sulfur compounds from a fuel, while copper-containing sorbents may be used to sorb H₂S.

The relative volumes of the beds 250, 252, 254 may be the same or different. In some embodiments, the relative volumes of the beds 250, 252, 254 may be set according to the amounts and/or types of contaminants in a fuel to be processed. For example, the hydrolysis bed 250 may occupy from about 5% to about 20%, such as about 10% of the total volume of the vessel 210 utilized for sulfur treatment, the first sorption bed 252 may occupy from about 0 to 40%, such as from about 1% to about 40%, about 20% to about 40%, or about 30% of the total volume of the vessel 210 utilized for sulfur treatment, and the second sorption bed 254 may occupy from about 40% to about 94%, such as from about 50% to about 70%, or about 60% of the total volume of the vessel 210 utilized for sulfur treatment.

In an alternative embodiment, the hydrolysis bed 250 may be located downstream of the first sorption bed 252. For example, the hydrolysis bed 250 may be located between the first sorption bed 252 and the second sorption bed 254. In this alternative embodiment, the first sorption bed 252 may remove at least a portion of the H₂S from the fuel before the fuel reaches the hydrolysis bed 250. This alternative bed configuration prevents or reduces H₂S interference with the hydrolysis reaction in the hydrolysis bed 250. The second sorption bed 254 is then used to remove one or more sulfur species from the fuel exiting the hydrolysis bed.

### Breakthrough Sulfur Detection

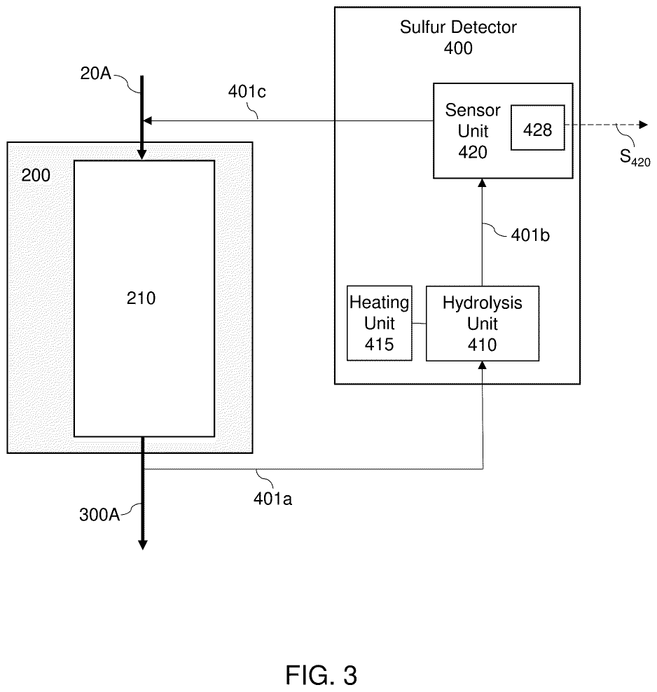

FIG. 3 illustrates an embodiment sulfur detector 400 that may be used with the desulfurization system 200 described above. The sulfur detector 400 and the desulfurization system 200 may be included in a fuel cell system, such as the SOFC system 10 in FIG. 1A. As illustrated in FIG. 3, the sulfur detector 400 may include a hydrolysis unit 410 (e.g., sulfur compound conversion unit) configured to hydrolyze a sulfur-containing compound, and a sensor unit 420 configured to detect the hydrolyzed sulfur-containing compound. The sulfur detector 400 may comprise a cost-effective and compact sulfur detection device.

In one embodiment, the above described hotbox 100 may be located in a power module 12 cabinet, and the above described desulfurization system 200 may be located in a fuel processing module (FPM) cabinet (for instance the cabinet for FPM 16 in FIG. 1B). In one embodiment, the FPM cabinet may be separate from one or more power module cabinets. In one embodiment, the FPM cabinet may be located on the same base (e.g., a concrete pad or a skid) as a plurality of power module cabinets. The sulfur detector 400 may be located in the FPM cabinet together with the desulfurization system 200. Alternatively, the sulfur detector 400 may be located downstream of the FPM cabinet (e.g., downstream of the desulfurization system 200), such as in a power module cabinet or another separate cabinet.

Natural gas may contain several different sulfur species including hydrogen sulfide (H₂S), carbonyl sulfide (COS), dimethyl sulfide (DMS), tetrahydrothiophene (THT), tertiary-butyl mercaptan (TBM), and disulfides, such as carbon disulfide (CS₂). Out of these, COS and CS₂ may be the most troublesome species to show early breakthrough in desulfurization media of the desulfurization system 200, as most desulfurization materials have limited capacity for these species. However, current commercial sulfur sensors are designed to detect hydrogen sulfide H₂S, and may not detect COS and CS₂ that have broken through the desulfurization system 200 into the hotboxes 100 of the power modules.

The embodiment sulfur detector 400 may be used, for example, as a breakthrough detector for breakthrough detection of sulfur-containing compounds such as COS and CS₂ at a fuel cell system level (e.g., for all power modules which receive fuel from the FPM). The sulfur detector 400 may utilize a simple mechanism of hydrolyzing sulfur-containing compounds such as COS and CS₂ into H₂S, and then detecting the H₂S. When the sulfur detector 400 detects H₂S, the sulfur detector 400 may generate a sulfur breakthrough signal to alert an operator to service the desulfurization system 200 (e.g., to replace the materials in the reaction vessels 210). Thus, the detector 400 may indirectly detect COS and CS₂ breakthrough by hydrolyzing these compounds to form H₂S, and then detecting the resulting H₂S.

As illustrated in FIG. 3, the sulfur detector 400 may include an inlet fuel line 401a, an internal fuel line 401b, a return fuel line 401c, a hydrolysis unit 410, a heating unit 415 and a sensor unit 420. The sulfur detector 400 may be connected to the fuel conduit 300A transporting desulfurized fuel (e.g., natural gas) away from the reaction vessel 210 (e.g., desulfurization cannister) of the desulfurization system 200. The sulfur detector 400 may be connected to the fuel conduit 300A by the inlet fuel line 401a. The inlet fuel line 401a may include, for example, a relatively narrow slipstream conduit (e.g., pipe, etc.). The inlet fuel line 401a may have a diameter, for example, in a range from 0.03 inches to 0.10 inches, such that only a small amount of fuel is diverted from the fuel conduit 300A to the inlet fuel line 401a.

The inlet fuel line 401a may transport a small amount of fuel from the fuel conduit 300A to the hydrolysis unit 410 of the sulfur detector 400. The hydrolysis unit 410 may include a vessel (e.g., capsule) containing a hydrolysis catalyst. The hydrolysis catalyst in the hydrolysis unit 410 may be substantially similar to the hydrolysis catalyst in the hydrolysis bed 250 described above and illustrated in FIG. 2B. In particular, the hydrolysis catalyst may include a sulfur hydrolysis catalyst configured to hydrolyze carbon-sulfur species, such as COS and/or CS₂, to produce hydrogen sulfide and carbon dioxide, using water present in the fuel. In one embodiment, the hydrolysis catalyst may include a commercially available alumina-based hydrolysis catalyst. Other types of hydrolysis catalysts in the hydrolysis unit 410 are within the contemplated scope of disclosure.

The hydrolysis unit 410 may operate on the principle of converting COS and CS₂ to H₂S by the following hydrolysis reaction mechanism in the presence of the hydrolysis catalyst:

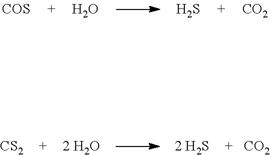

If the hydrolysis unit 410 includes a hydrolysis catalyst which operates above room temperature, then the sulfur detector 400 may also include a heating unit 415 for heating the hydrolysis catalyst in the hydrolysis unit 410. The heating unit 415 may heat the heating catalyst in the hydrolysis catalyst to a temperature in a range from 80° C. to 140° C., such as 100° C. to 120° C. The heating unit 415 may be located adjacent the hydrolysis unit 410 in the sulfur detector 400. The heating unit 415 may include, for example, a resistance heater. In one embodiment, the resistance heater may comprise an electrical tape or another resistor material wrapped around the hydrolysis unit 410, and electrically connected to a power supply, such as a current or voltage source. For example, the power supply may comprise a 24V power supply configured to provide a relatively small amount of power to the electrical tape to heat the small amount of hydrolysis catalyst present in the hydrolysis unit 410. Other types of heating units 415 are within the contemplated scope of disclosure. For instance, if the sulfur detector is located nearby a hotbox 100, heating unit 415 could comprise a heat exchanger that transfers heat from hotbox exhaust from conduit 304C to the hydrolysis catalyst in the hydrolysis unit 410. Alternatively, if the hydrolysis catalyst operates at room temperature, the heating unit 415 may be omitted.

The fuel may be transported from the hydrolysis unit 410 to the sensor unit 420 by an internal fuel line 401b. The internal fuel line 401b may be substantially the same as the inlet fuel line 401a. The sensor unit 420 may detect the presence of H₂S in the fuel provided from the internal fuel line 401b. The sensor unit 420 may detect the presence of H₂S at the parts per billion level. The sensor unit 420 may also measure a level of the H₂S in the fuel.

The sensor unit 420 may include, for example, a resistance change sensor, an electrochemical sensor, a microelectromechanical system (MEMS) sensor, an optical sensor, an acoustic sensor, a gas chromatography-ion mobility spectrometry (GC-IMS) device with a H₂S detection channel, etc. Other types of sensors are within the contemplated scope of disclosure. The sensor unit 420 may include a sensing circuit 428 (e.g., electrical circuit). The sensing circuit 428 may sense the presence of H₂S in the fuel and generate a sensing signal S₄₂₀ indicating that H₂S has been detected in the fuel. Fuel that has passed through the sensor unit 420 may be transported from the sensor unit 420 to the fuel source conduit 20A by the return fuel line 401c. The return fuel line 401c may be substantially the same as the inlet fuel line 401a and the internal fuel line 401b.

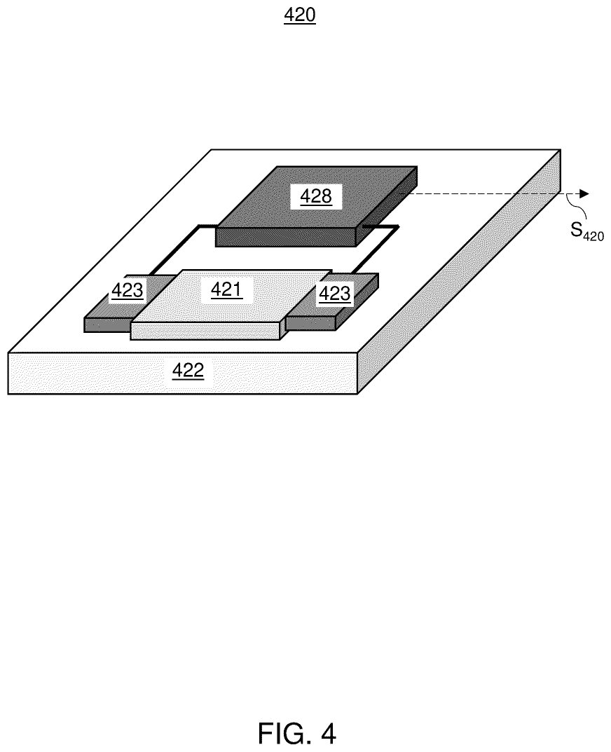

FIG. 4 illustrates an exemplary sensor unit 420 that may be utilized in the sulfur detector 400, according to one or more embodiments of the present disclosure. As illustrated in FIG. 4, the sensor unit 420 may include sensing element 421 on a substrate 422 (e.g., a printed circuit board (PCB), etc.). The sensing element 421 may include a sensing material that may interact with H₂S (target gas) causing a change in electrical resistance of the sensing material. In one embodiment, the sensing element may include a metal oxide material, such as tin oxide, or another wide bandgap semiconductor material, which adsorbs H₂S and whose resistivity changes as a function of H₂S adsorption. The sensor unit 420 may also include a pair of electrodes 423 (e.g., metal electrodes) connected to the sensing element 421 on opposite sides of the of the sensing element 421.

The sensing circuit 428 may be electrically coupled to the pair of electrodes 423. The sensing circuit 428 may cause a small sensing voltage or current to be applied across the sensing element 421 using the electrodes 423. The sensing circuit 428 may monitor the sensing voltage or current and detect a change in electrical resistance of the sensing material (i.e., an increase or decrease in voltage or current flowing between the electrodes 423) in the sensing element 421 caused by an interaction (e.g., absorption, adsorption, desorption, etc.) of the H₂S (target gas) with the sensing material.

The sensing circuit 428 may also perform, for example, signal conditioning, signal processing and data display. In particular, the sensing circuit 428 may condition a signal generated by the electrodes 423 by amplifying it to make the it strong enough for processing and filtering out noise to improve accuracy. The sensing circuit 428 may also convert the conditioned signal from an analog form (continuous) to a digital signal using an analog-to-digital converter (ADC). The sensing circuit 428 may include, for example, a microcontroller or processor that interprets the digital data by correlating it to the presence of H₂S. The sensor circuit 428 may also include a transmitter (e.g., wired or wireless transmitter) for transmitting the sensing signal S₄₂₀ to another device such as a controller (e.g., a local or system controller), a display unit, visual alarm device (e.g., flashing light), audible alarm device, etc.

While a resistance change sensor unit 420 is described above, other types of sensor units may also be used. An electrochemical sensor may include an electrolyte located between working and reference electrodes. H₂S molecules undergo an oxidation reaction at the working electrode, in which the hydrogen sulfide molecules lose electrons and are oxidized to sulfate ions (HSO⁴⁻). During the oxidation of hydrogen sulfide, electrons are released and flow through the sensor's circuit, resulting in the generation of an electric current. The magnitude of this current is proportional to the hydrogen sulfide concentration. The current is measured and converted to H₂S concentration.

An inertial or bifurcation MEMS sensor may include one or more functionalized cantilevers located above electrodes or in contact with electrodes. The cantilever mass, bending, vibration orbit or vibration frequency changes based on adsorption of the H₂S molecules to the functional groups on the cantilevers. This change affects the current or voltage flowing between the electrodes.

An optical sensor may include a MEMS optical sensor containing a functionalized cantilever or membrane, a laser and a photodetector. The laser emits a laser beam onto the cantilever or membrane, and the reflected laser beam is detected by the photodetector. If H₂S molecules are adsorbed to the cantilever or membrane, then its bending, vibration orbit or vibration frequency changes based on adsorption of the H₂S molecules to the functional groups on the cantilever or membrane. This change affects the detected laser beam which is reflected from the cantilever or membrane. Alternatively, the optical sensor may include a color change sensor, which changes color based on adsorption of the H₂S molecules to a functionalized surface. A spectrometer detects the color change to detect the presence of the H₂S molecules. An acoustic sensor may include a MEMS acoustic sensor in which the sound of the sensor changes upon detection of the H₂S molecules.

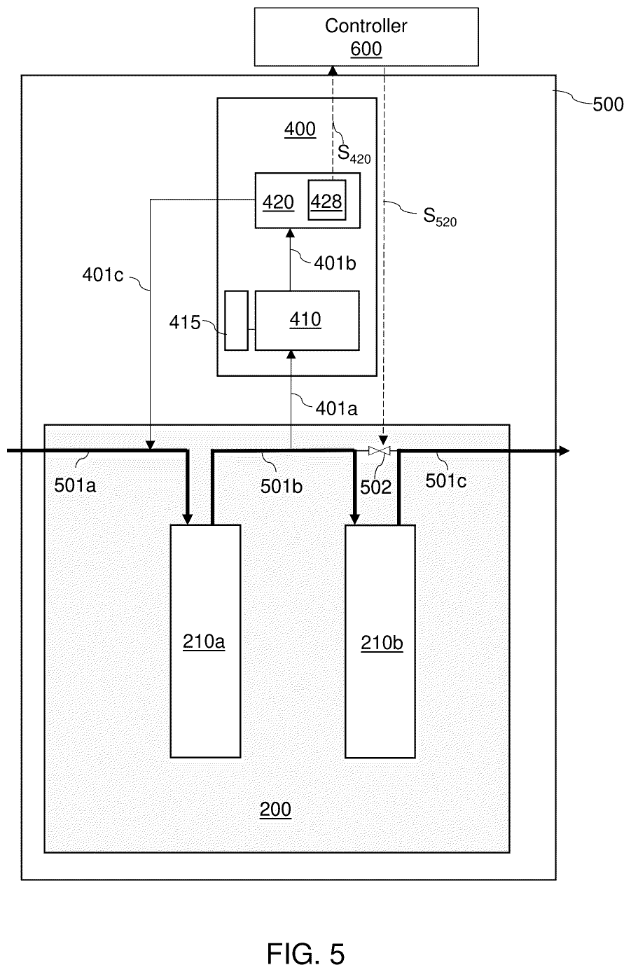

FIG. 5 illustrates an alternative configuration of the sulfur detector 400 integrated with the desulfurization system 200 in a FPM cabinet 500 which is also shown in FIG. 1B. As illustrated in FIG. 5, the desulfurization system 200 includes a plurality of the reaction vessels 210 (e.g., desulfurization cannisters) including at least one primary vessel 210a and a backup vessel 210b fluidly connected in series. The desulfurization system 200 may also include an internal inlet fuel line 501a transporting fuel to an inlet of the primary vessel 210a. The desulfurization system 200 may also include an internal inlet/outlet fuel line 501b transporting fuel from an outlet of the at least one primary vessel 210a to an inlet of the backup vessel 210b. While one primary vessel 210a is illustrated for simplicity, it should be noted that plural primary vessels 210a may fluidly connected in series by the internal inlet/outlet fuel line 501b. The desulfurization system 200 may also include an internal outlet fuel line 501c transporting fuel from an outlet of the backup vessel 210b, and a bypass valve 502 fluidly connecting the internal inlet/outlet fuel line 501b to the internal outlet fuel line 501c.

As further illustrated in FIG. 5, the sulfur detector 400 in the alternative configuration may be substantially the same as the sulfur detector 400 in FIG. 3. However, in the alternative configuration in FIG. 5, the inlet fuel line 401a (e.g., slipstream line) may be connected to the internal inlet/outlet fuel line 501b in the desulfurization system 200. In addition, the return fuel line 401c may transport fuel from the sulfur detector 400 to the internal inlet fuel line 501a in the desulfurization system 200.

During steady-state operation of the FPM located in the FPM cabinet 500, the bypass valve 502 is open to permit the desulfurized fuel exiting the at least one primary vessel 210a via the internal inlet/outlet fuel line 501b to bypass the backup vessel 210b and to flow directly form the internal inlet/outlet fuel line 501b to the internal outlet fuel line 501c and out of the FPM cabinet 500 to the hotboxes 100 of the power modules 12. If the sulfur detector 400 detects sulfur compound slippage past the at least one primary vessel 210a into the internal inlet/outlet fuel line 501b, the sensor circuit 428 transmits the sulfur sensing signal S₄₂₀ to the controller (e.g., a local or system controller) 600. In response to receiving the sulfur sensing signal S₄₂₀ indicating the breakthrough of sulfur species into the internal inlet/outlet fuel line 501b, the controller 600 sends a control signal S₅₂₀ to the bypass valve 502 to close the bypass valve 502. The controller 600 also sends a signal to control personnel to change the sorption bed material in the at least one primary vessel 210a. The closure of the bypass valve causes the fuel to pass through the backup vessel 210b to remove the sulfur species from the fuel before the fuel is provided to the hotboxes 100 via line 501c. Once the sorption bed material in the at least one primary vessel 210a is replaced, the bypass valve 502 is opened and the steady-state operation of the FPM resumes.

Thus, the method of operating the system of FIGS. 1A, 1B and 5 includes providing the fuel to a desulfurization system 400 comprising at least one primary reaction vessel 210a and a backup reaction vessel 210b configured to desulfurize the fuel; and detecting the hydrogen sulfide in the desulfurized fuel using the hydrogen sulfide sensor unit 420 of the sulfur detector 400. The desulfurized fuel is provided from the at least one primary reaction vessel 210a to at least one fuel cell stack 102 while bypassing the backup reaction vessel 210b through the open bypass valve 502 if the hydrogen sulfide is not detected in the desulfurized fuel. In contrast, the desulfurized fuel is provided from the at least one primary reaction vessel 210a to the at least one fuel cell stack 102 through the backup reaction vessel 210b by closing the bypass valve 502 if the hydrogen sulfide is detected in the desulfurized fuel.

The embodiment sulfur detector 400 may reduce system cost by providing a carbon and sulfur containing compound (e.g., COS and/or CS₂) breakthrough detection using an inexpensive H₂S sensor. The sulfur detector 400 may facilitate early detection of carbon sulfur compound slippage and permit proactive intervention by converting carbon sulfur compounds into H₂S using a hydrolysis catalyst. Furthermore, the compact design of the sulfur detector 400 may have a smaller footprint allowing for easier integration into existing infrastructure.

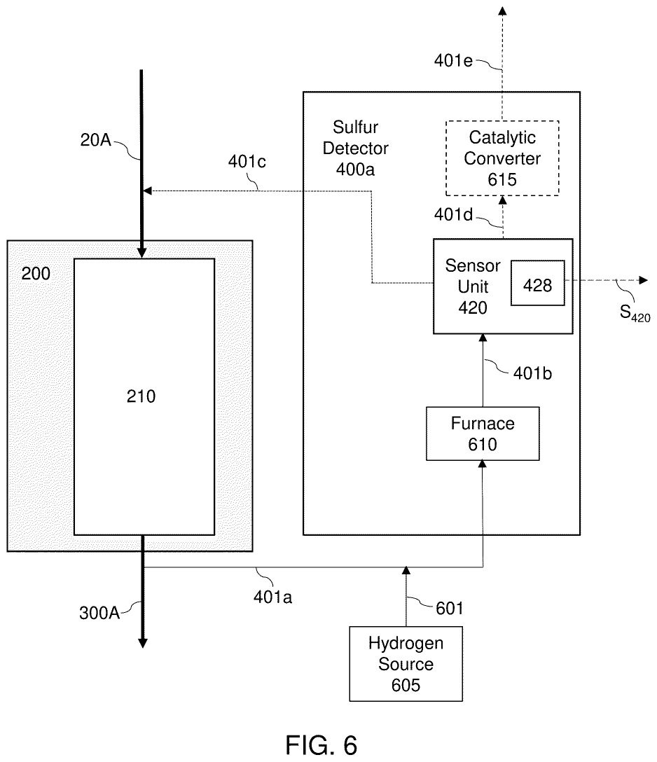

FIG. 6 illustrates an alternative configuration of the sulfur detector 400a (e.g., breakthrough detector) which may be used with the desulfurization system 200 described above. The alternative sulfur detector 400a having the alternative configuration shown in FIG. 6 may be substantially similar to the sulfur detector 400 shown in FIG. 3. However, unlike the sulfur detector 400 shown in FIG. 3 which may include a hydrolysis unit 410 and heating unit 415, the alternative sulfur detector 400a in FIG. 6 may include a furnace 610 (e.g., sulfur compound conversion unit) for converting sulfur compounds other than H₂S in the fuel inlet stream to H₂S by a high temperature reaction with added hydrogen gas. The hydrolysis unit 410 and the heating unit 415 may be omitted from the alternative sulfur detector 400a.

The alternative sulfur detector 400a shown in FIG. 6 may also include an optional catalytic converter 615 (e.g., hydrocarbon and volatile organic compound (VOC) emission catalytic converter) and an optional analyzed stream line 401d for transporting analyzed fuel from the outlet of the sensor unit 420 to an inlet of the catalytic converter 615. The sulfur detector 400a may also include an optional vent line 401e for venting exhaust (e.g., water vapor and carbon dioxide) from the outlet of the catalytic converter 615 to the atmosphere. Alternatively, the catalytic converter 615, the analyzed stream line 401d and the vent line 401e may be omitted. Instead, the above described return fuel line 401c may fluidly connect the outlet of the sensor unit 420 to the fuel source conduit 20A, as described above with respect to FIG. 3.

In particular, in contrast to the sulfur detector 400 shown in FIG. 3 which may use catalytic hydrolysis to convert carbon and sulfur containing compounds, such as COS and/or CS₂, to H₂S for detection, the alternative sulfur detector 400a shown in FIG. 6 may perform a high temperature conversion of carbon and sulfur containing compounds in the fuel inlet stream to H₂S in the presence of additional hydrogen gas. Thus, the alternative sulfur detector 400a shown in FIG. 6 may react at least one carbon and sulfur containing compound with hydrogen gas to form H₂S for detection in the sensor unit 420 instead of using a hydrolysis process to convert the carbon and sulfur containing compounds to H₂S for detection.

The sulfur detector 400a in FIG. 6 may perform quantification and analysis of total sulfur in a sample gas (e.g., the desulfurized fuel inlet stream for a fuel cell system, such as desulfurized biogas, desulfurized natural gas, etc.) at low levels, such as less than 100 parts per billion by volume (ppbV), via conversion of all or substantially all sulfur compounds other than H₂S in the fuel inlet stream to H₂S by reaction with hydrogen gas, followed by a H₂S measurement using the sensor unit 420 (e.g., the hydrogen sulfide detector described above). By converting all sulfur compounds other than H₂S, such as carbon and sulfur containing compounds (e.g., COS and/or CS₂), to H₂S, the alternative sulfur detector 400a shown in FIG. 6 may measure total sulfur content, as opposed to measuring different sulfur species, thereby addressing the limitations in existing analyzers that either measure total sulfur at higher detection limits (e.g., greater than 30 ppbV) or detect only a limited number of sulfur species (e.g., 3 to 5 species) at lower limits (e.g., 5 to 10 ppbV). By achieving high conversion efficiency (such as 99% or greater, including 99.9% to 100%) of sulfur compounds other than H₂S to H₂S, the total sulfur concentration in the desulfurized fuel inlet stream may be determined using commercially available, low-cost H₂S sensors, which are capable of detecting H₂S at 1-10 ppbV levels.

As illustrated in FIG. 6, the hydrogen gas may be added to the desulfurized fuel inlet stream provided via the inlet fuel line (e.g., slipstream conduit) 401a that fluidly connects the fuel conduit 300A to the alternative sulfur detector 400a. The desulfurized fuel inlet stream in the inlet fuel line 401a may have a bulk composition including primarily hydrocarbons (e.g., methane in natural gas or a mixture of methane and carbon dioxide in biogas), with various sulfur species at low concentrations (e.g., less than 100 ppbV total sulfur). The sulfur content in the fuel inlet stream may include various species, such as H₂S, mercaptans, thiophenes, dimethyl sulfide, carbonyl sulfide, carbon disulfide, etc. The desulfurized fuel inlet stream in the inlet fuel line 401a may be provided at a flow rate of 0.01 to 1 standard liters per minute (SLM), such as 0.1 to 0.5 SLM and a pressure of about 5 to 50 pounds per square inch gauge (psig), such as 15 to 30 psig.

The fuel inlet stream and the hydrogen gas are provided to the furnace 610 either separately or as a gas mixture via a hydrogen supply line 601. In one embodiment, the hydrogen supply line 601 may be indirectly fluidly connected to the furnace 610 via the inlet fuel line 401a. In this embodiment the hydrogen supply line 601 includes an outlet that is fluidly connected to the fuel inlet line 401a to provide a mixture of the fuel inlet stream and the hydrogen gas to the furnace 610. Alternatively, the outlet of the hydrogen supply line 601 may be directly fluidly connected to the furnace 610 rather than to the inlet fuel line 401a, to separately provide the fuel inlet stream and the hydrogen gas to the furnace 610. The inlet hydrogen supply line 601 may be fluidly connected to a hydrogen source 605, such as a hydrogen tank, hydrogen supply system (e.g., an electrolyzer or a chemical hydrogen generation system), etc. The hydrogen gas may be provided via the hydrogen supply line 601 at a flow rate of about 0.05 to 2 SLM, such 0.2 to 0.5 SLM and a pressure of about 5 to 50 psig, such as 15 to 30 psig. In one embodiment, the flow rate of the hydrogen gas (e.g., H₂) is higher than the flow rate of the fuel inlet stream provided to the sulfur detector 400a.

The furnace 610 may comprise, for example, an electric furnace, a gas fired furnace or any other suitable heater. The interior of the furnace 610 may optionally include a catalyst which facilitates a high temperature reaction of sulfur containing compounds with hydrogen gas to form H₂S and at least one additional gas that excludes sulfur. Alternatively, the catalyst may be omitted to decrease the cost of the furnace 610. The interior of the furnace 610 may also include flow structures to ensure adequate mixing of the fuel inlet stream gas and the hydrogen gas.

The furnace 610 may heat the fuel inlet stream and the hydrogen gas to convert the sulfur compounds other than H₂S in the fuel inlet stream to H₂S. The furnace 610 may be operated at a relatively high temperature, such as 800° C. or greater, including 800° C. to 1100° C., e.g., 900° C. to 1000° C., to facilitate the conversion of substantially all sulfur compounds other than H₂S in the fuel inlet stream to H₂S and at least one additional gas that excludes sulfur in the presence of the H₂. For example, carbonyl sulfide and carbon disulfide may react with hydrogen gas at the relatively high temperature to produce hydrogen sulfide and at least one additional carbon containing gas:

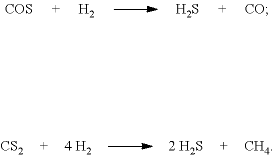

Thus, carbonyl sulfide and carbon disulfide may react with the added hydrogen gas to form hydrogen sulfide gas and either carbon monoxide or methane gas, respectively. This high-temperature hydrogen reaction process may achieve a carbon and sulfur containing compound conversion efficiency of 99% or greater (e.g., 99.9% to 100% by volume). The furnace 610 may produce an output stream with a sulfur compound content that is at least 99% H₂S, such as 99.9% to 100% by volume and a balance of any unconverted sulfur species.

The output stream from the furnace 610 may be transported to the above described sensor unit 420 by the internal fuel line 401b. The output stream may exit the furnace 610 in the internal fuel line 401b at a flow rate that is the same as or lower than the flow rate of the gases entering the furnace 610. In embodiments where the sensor unit 420 includes a GC-IMS, the sensor unit 420 may provide a total sulfur measurement in addition to or instead of compound-specific data. The sensor unit 420 may measure a concentration of H₂S in the mixture of the desulfurized fuel inlet stream and any unreacted hydrogen gas in the internal fuel line 401b, which may correspond to a total sulfur content of the desulfurized fuel inlet stream in the inlet fuel line 401a due to the high conversion efficiency of the hydrogen reaction process in the furnace 610.

The analyzed fuel inlet stream may be transported from the sensor unit 420 by internal fuel line 401d to the catalytic converter 615. The catalytic converter 615 is configured to oxidize or otherwise process hydrocarbons and volatile organic compounds (VOCs) in the analyzed fuel inlet stream. The catalytic converter 615 may include, for example, an oxidation catalyst such as platinum, palladium, ruthenium, etc. The oxidation catalyst may be supported on a high-surface area metal oxide support, such as an alumina support. The catalytic converter 615 may produce an exhaust including primarily water vapor and carbon dioxide.

The exhaust may exit the catalytic converter 615 at a flow rate of less than the flow rate of the gases entering the furnace 610, such as 0.05 to 0.75 SLM, and a pressure less than atmospheric pressure. The exhaust may be vented to the atmosphere via vent line 401e or sequestered for storage or suitable use. Alternatively, the catalytic converter 615, the analyzed stream line 401d and the vent line 401e may be omitted. Instead, the analyzed fuel inlet stream may be transported from the sensor unit 420 to the fuel source conduit 20A via the return fuel line 401c, as described above with respect to FIG. 3.

As noted above, the sensor unit 420 may transmit the sulfur sensing signal S₄₂₀ to a controller 600 (e.g., system controller 225). The sulfur sensing signal S₄₂₀ from the sensor unit 420 may indicate the total sulfur concentration in the fuel (i.e., the desulfurized fuel inlet stream) in the fuel conduit 300A. If the total sulfur concentration indicated by the sulfur sensing signal S₄₂₀ exceeds a predetermined threshold (e.g., 10 ppbV), the controller may initiate corrective actions, such as activating a bypass valve (e.g., bypass valve 502), alerting personnel for sorbent replacement in reaction vessels 210, or adjusting fuel flow to prevent sulfur ingress to the fuel cell stack 102.

The alternative sulfur detector 400a shown in FIG. 6 may be used, for example, at sites where gas desulfurization is centralized, resulting in a single point of failure for the site, making accurate sulfur quantification important. For these sites, it may be helpful to have the ability to measure very low levels of sulfur with accuracy, to prevent failures that might have a sitewide impact due to sulfur breakthrough. The alterative sulfur detector 400a may also be utilized, for example, at biogas sites where various sulfur species can vary from day to day, season to season, or as a single event due to poor digester performance that can spike any sulfur species rapidly in one day. The alternative sulfur detector 400a may be less expensive than the sulfur detector 400 because it preferably omits the relatively expensive hydrolysis unit 410 containing a high cost hydrolysis catalyst.

The preceding description of the disclosed aspects is provided to enable any person skilled in the art to make or use the present invention. Various modifications to these aspects will be readily apparent to those skilled in the art, and the generic principles defined herein may be applied to other aspects without departing from the scope of the invention. Thus, the present invention is not intended to be limited to the aspects shown herein but is to be accorded the widest scope consistent with the principles and novel features disclosed herein.

What is claimed is:

1. A sulfur detector, comprising:
a sulfur compound conversion unit configured to convert a sulfur-containing compound into hydrogen sulfide (H₂S); and
a sensor unit configured to detect the H₂S.

2. The sulfur detector of claim 1, wherein the sulfur compound conversion unit comprises a hydrolysis unit configured to hydrolyze the sulfur-containing compound into the H₂S.

3. The sulfur detector of claim 2, wherein:
the sulfur-containing compound comprises at least one of carbonyl sulfide or carbon disulfide; and
the hydrolysis unit comprises a hydrolysis catalyst configured to hydrolyze the sulfur-containing compound to produce the hydrogen sulfide and carbon dioxide.

4. The sulfur detector of claim 3, further comprising a heating unit configured to heat the hydrolysis catalyst, wherein the hydrolysis catalyst comprises an alumina-based catalyst.

5. The sulfur detector of claim 4, wherein the sensor unit comprises a resistance change sensor comprising:
a sensing element comprising a sensing material configured to adsorb or absorb the hydrogen sulfide;
electrodes connected to the sensing element; and
a sensing circuit configured to detect a change in electrical resistance of the sensing element due to absorption or adsorption of the hydrogen sulfide on a surface of the sensing element, wherein the sensing circuit is configured to generate a sensing signal indicating the change in electrical resistance of the sensing element in response to adsorption or absorption of the hydrogen sulfide on the surface of the sensing element.

6. The sulfur detector of claim 1, wherein:
the sulfur compound conversion unit comprises a furnace; and
the sulfur-containing compound comprises at least one of a mercaptan, a thiophene, dimethyl sulfide (DMS), carbonyl sulfide (COS), or carbon disulfide (CS₂).

7. The sulfur detector of claim 6, further comprising a hydrogen supply line that is fluidly connected to the furnace, wherein:
the hydrogen supply line is configured to provide hydrogen gas to the furnace from a hydrogen source; and
the furnace is configured to react the sulfur-containing compound with the hydrogen gas to form the H₂S and at least one carbon-containing gas at a temperature of at least 800° C.

8. The sulfur detector of claim 7, wherein the furnace is configured to react at least one of the COS or the CS₂ with the hydrogen gas to form the H₂S and at least one of carbon monoxide or methane, respectively.

9. The sulfur detector of claim 7, further comprising a catalytic converter fluidly connected to an outlet of the sensor unit and configured to convert at least one organic compound in an output stream provided from the outlet of the sensor unit into water vapor and carbon dioxide.

10. The sulfur detector of claim 1, wherein the sensor unit comprises a resistance change sensor, an electrochemical sensor, a microelectromechanical system (MEMS) sensor, an optical sensor, an acoustic sensor or a gas chromatography-ion mobility spectrometry (GC-IMS) device.

11. A system comprising:
a desulfurization system comprising a plurality of reaction vessels configured to desulfurize a fuel;
a fuel cell power module comprising fuel cells;
a fuel conduit fluidly connecting the desulfurization system to the fuel cell power module, and configured to transport the desulfurized fuel from the desulfurization system to the fuel cell power module; and
the sulfur detector of claim 1 fluidly connected to the fuel conduit and configured to convert the sulfur-containing compound into the H₂S, and to detect the H₂S.

12. The system of claim 11, wherein the fuel conduit comprises:
an internal inlet/outlet fuel line fluidly connecting an outlet of at least one primary vessel of the plurality of reaction vessels to an inlet of a backup vessel of the plurality of reaction vessels, and configured to transport the fuel from the outlet of the at least one primary vessel to the inlet of the backup vessel;
an internal outlet fuel line fluidly connected to an outlet of the backup vessel; and
a bypass valve fluidly connecting the internal inlet/outlet fuel line to the internal outlet fuel line and bypassing the backup vessel.

13. The system of claim 12, further comprising:
a fuel source configured to provide the fuel to the desulfurization system;
a slipstream line fluidly connecting the fuel conduit to the sulfur detector, and configured to provide the desulfurized fuel from the fuel conduit to the sulfur detector;
a return line configured to transport the desulfurized fuel from the sulfur detector to the desulfurization system; and
a controller configured to:
receive a sulfur sensing signal from the sulfur detector indicating breakthrough of the sulfur-containing compound into the internal inlet/outlet fuel line; and
send a control signal to the bypass valve to close the bypass valve to provide the fuel into the backup vessel in response to receiving the sulfur sensing signal.

14. A method of detecting a sulfur-containing compound in a fuel, comprising:
converting the sulfur-containing compound in the fuel into hydrogen sulfide (H₂S); and
detecting the H₂S.

15. The method of claim 14, wherein:
the converting the sulfur-containing compound comprises hydrolyzing at least one of carbonyl sulfide (COS) or carbon disulfide (CS₂) using a hydrolysis catalyst to produce the H₂S and carbon dioxide; and
the detecting the H₂S comprises detecting the H₂S using a hydrogen sulfide sensor.

16. The method of claim 15, further comprising heating the hydrolysis catalyst.

17. The method of claim 14, wherein:
the converting the sulfur-containing compound comprises reacting a sulfur-containing compound other than H₂S with hydrogen gas at a temperature of at least 800° C. to produce the H₂S and at least one carbon-containing gas; and
the detecting the H₂S comprises detecting the H₂S using a hydrogen sulfide sensor.

18. The method of claim 17, wherein:
the sulfur-containing compound comprises at least one of COS or CS₂; and
the at least one carbon-containing gas comprises at least one of carbon monoxide or methane, respectively.

19. The method of claim 14, further comprising:
providing the fuel to a desulfurization system comprising at least one primary reaction vessel and a backup reaction vessel configured to desulfurize the fuel;
providing the desulfurized fuel from the at least one primary reaction vessel to at least one fuel cell stack while bypassing the backup reaction vessel if the H₂S is not detected in the desulfurized fuel; and
providing the desulfurized fuel from the at least one primary reaction vessel to the at least one fuel cell stack through the backup reaction vessel if the H₂S is detected in the desulfurized fuel.

20. The method of claim 14, wherein the H₂S is detected by a resistance change sensor, an electrochemical sensor, a microelectromechanical system (MEMS) sensor, an optical sensor, an acoustic sensor or a gas chromatography-ion mobility spectrometry (GC-IMS) device.
